(19) United States
(12) Patent Application Publication
(10) Pub. No.: US 2005/0055101 A1
(43) Pub. Date: Mar. 10, 2005

(54) Endoprosthesis of the knee and/or other joints

(76) Inventor: Dimitrios Sifneos, Paros Cyclades (GB)

Correspondence Address: DAVID A. GUERRA, 933 OLANDER WAY SOUTH, SUITE 3, SOUTH PASADENA, FL, 33707, US

(21) Appl. No.: 10/497,637

(22) PCT Filed: Dec. 9, 2002
(86) PCT No.: PCT/GR02/00065

(30) Foreign Application Priority Data
Dec. 11, 2001 (GR) 20010100585

(51) Int. Cl. A61F 2/38; A61F 2/30

(52) U.S. Cl. 623/20.32; 623/22.13; 623/23.41

(57) ABSTRACT

The principle of this endoprosthesis of the knee can also be used in other joints, that require a bearing during movement of the parts, with the aim of reducing wear. This endoprosthesis has improved self-lubrication mechanisms, the ability to filter the particles from the debris produced by the moving parts, and a new viscoelastic behavior under loading which reduce the transmitted forces. This has been achieved with the use of compressible materials (13) and mechanisms [for example between the fixed bearing (25) and the tibial component (24)], allowing the endoprosthesis to have compressibility under loading, which allows it also to receive or create chambers (14) with an exit (26) to the surface articulating with the femoral condyles (15 ). These chambers (14) can be compressed (13a) and decompressed (13b), accumulate and spurt out (16) synovial fluid, from and to the articulating surfaces. The movement of the synovial fluid and gases from and to (16-19) the exit (26) openings creates viscoelasticity, and improves the lubrication of the moving parts. Simultaneously, permeable filter (21) materials interpolate to the synovial fluid flow (16-19) trapping the debris.

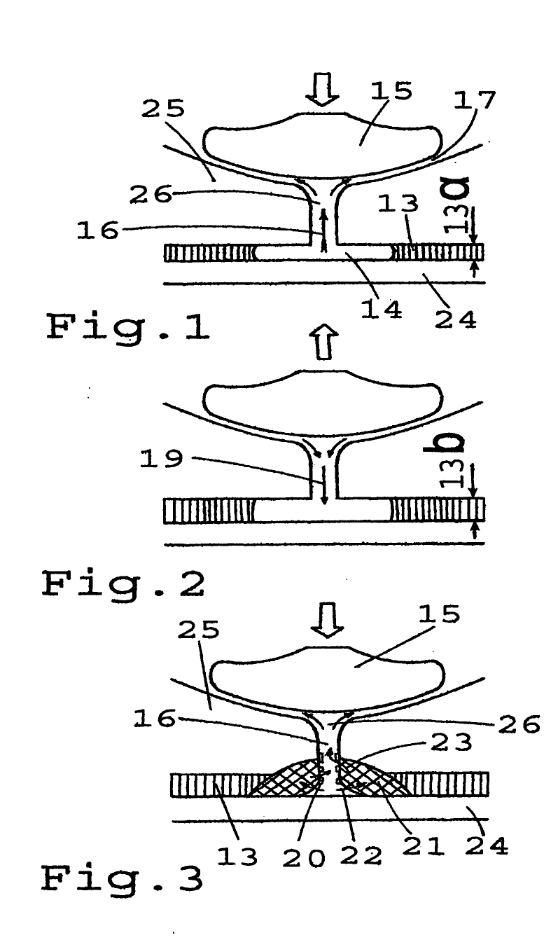

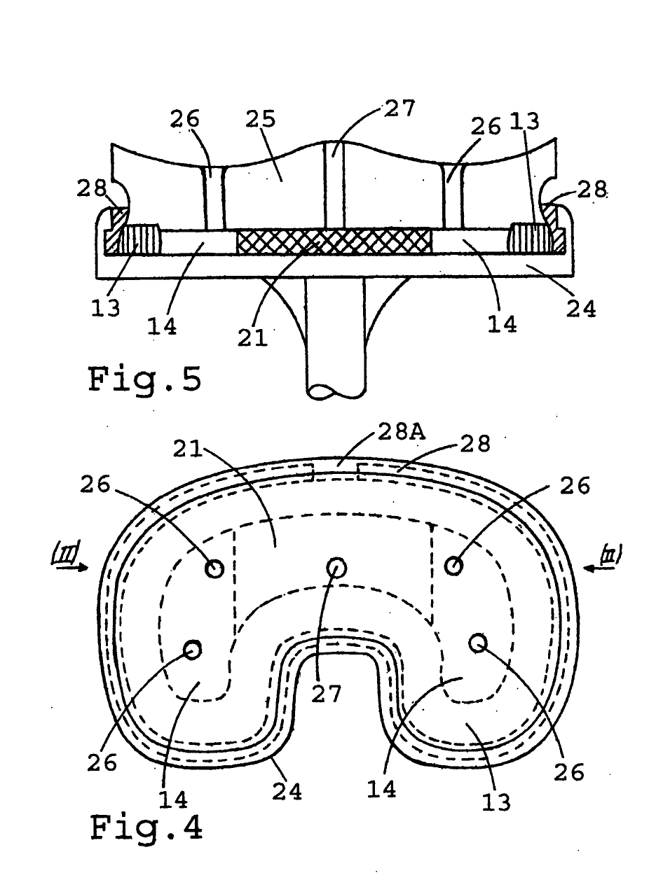

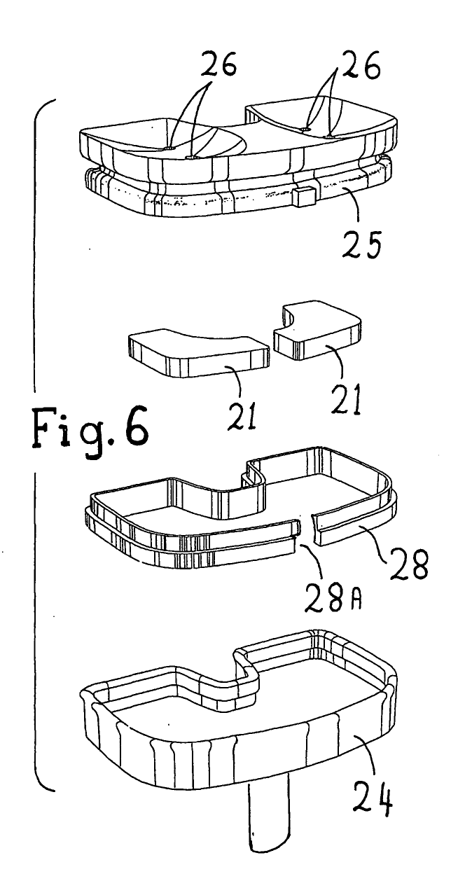

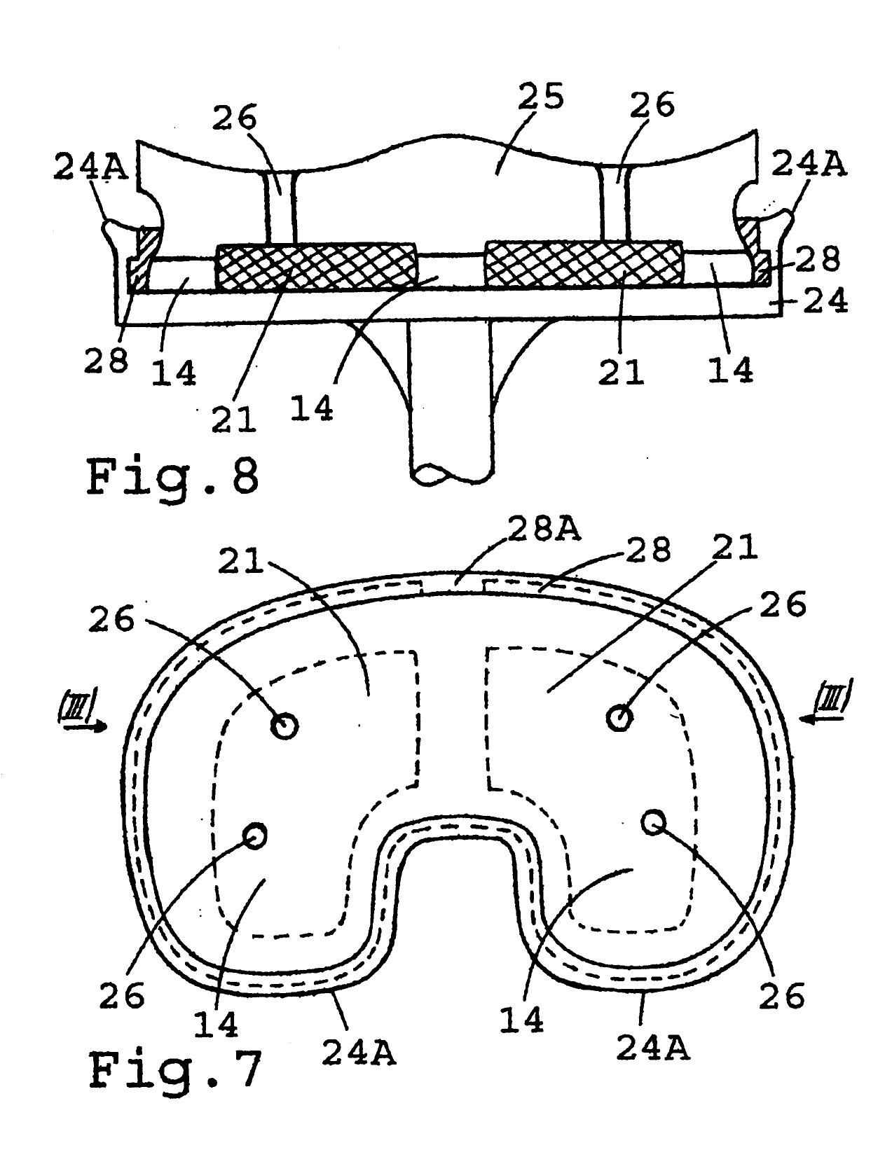

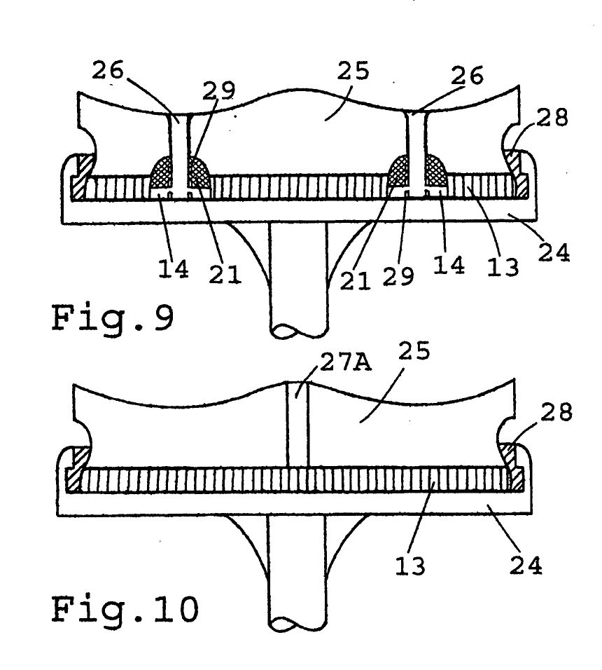

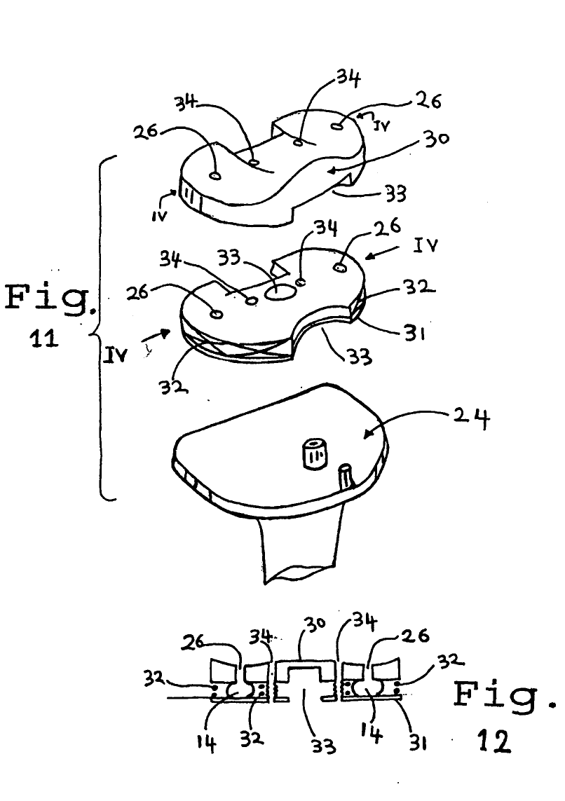

The invention concerns a endoprosthesis of the knee and/or other joints, where the implant or platform, mobile or fixed, of polyethylene or other material is used.

The standard endoprosthesis of a knee joint consists of three parts,
1. the femoral component,
2. the tibial component
3. & an implant made of polyethylene or other material, which can be either fixed on to the tibial component, or mobile in relation to the tibial component.

Subsequently, the description, when the implant platform is fixed on the tibial component will be called “the fixed bearing”, and when the implant platform moves on the tibial component will be called “mobile bearing”. The superior surface of the fixed bearing or mobile bearing articulates with the metal or other material femoral component, and the inferior surface with the tibial component. It is known that the physiological articular cartilage of the joints generally (and particularly the knee joint which receives extensive forces) is protected from wear and injuries by the following three properties:
1. it has the ability to absorb forces and compressive loads that are applied in an elastic and viscoelastic way.
2. the ability of the articular cartilage to adapt its surfaces by conforming itself after a period of static loading.
3. The continuous lubrication of the synovial fluid which is in the joint space and in the articular cartilage.

There are 4 theories how this lubrication occurs: boundary lubrication, hydrodynamic lubrication, weeping lubrication, and elastohydrodynamic lubrication. (Journal of Orthopedic and Sports Physical Therapy, 0196-611/82/0304-0186 (volume 13, number 4).

The viscoelastic absorption of the forces are exerted on to the articular cartilage and are due to combined factors.
A—progressive entry and exit of the synovial fluid within the substance of the articular cartilage.
B—the elastic absorption of the forces from the collagenous fibres, the proteoglycan network, and solid fibres of the articular cartilage.

This invention refers to an endoprosthesis for the knee and/or other joints that use a fixed or mobile bearing, and components covering the articular surface of the bones.

This invention is characterized by viscoelastic behavior during compression (as in loading) and during decompression (as in unloading), by integral chambers in the components that consist of, chambers which communicate with the joint space through small openings or holes. It is also characterized by the possibility of the chambers ability to aspirate and accumulate synovial fluid and gases during unloading and extrude fluid and gases to the joint space during loading. Another characteristic is that it has materials which are permeable to the synovial fluid. These materials interpolate to the synovial fluid flow and filter it from the wear debris which circulates in the synovial fluid of the endoprosthesis.

Until now the fixed or mobile bearings that are being used in total knee endoprosthesis, or other joint endoprostheses, have mainly been made from ultra high molecular weight polyethylene or other material such as ceramic. The manufactured bearings and their connection with the tibial component have neither the ability nor the function to make use of the synovial fluid for an effective lubrication. They do not have the ability to filter the debris particles, nor to progressively absorb the forces that are exerted during knee movement.

Another element is that the bearings either mobile or fixed to the tibial component cannot adapt their shape and position in relation to the anteroposterior and transverse axis, which adaptation is relevant to the direction of load received. As a result of this, there is increased wear of the bearings and moving surfaces especially of the polyethylene, because the surfaces move with increased frictional forces that cause scraping, delamination, pitting, and fatigue.

The bearings do not have the above-named properties and the surfaces are less conforming, and therefore there is unequal distribution of load. These features contribute significantly to the potential failure of the endoprosthesis, instead of the expected successful result from such an operation.

In October 2001, a new prosthesis was described as a patent application with an elastic material between the inferior and superior parts of its mobile bearing body, (pat.GB2348373)—another description (GB2348373) is of a tibial prosthesis component with an elastic material between the upper part of the metal tibial component that comes into contact with the mobile bearing and the metallic undersurface sitting on the tibial bone. Neither of these applications offer any potential for the lubrication of the surfaces and therefore no reduction in the damage from wear, such as scraping and delamination etc. Neither do they have the ability to filter the debris, nor a progressive viscoelastic absorption of the forces or adaptation, and specifically the description of the mobile bearing with only one compressible material between the upper and lower part of its body may be unsafe and be difficult for the bearing to have manufacturing bonding.

In contrast to the above, this invention aims to produce an endoprosthesis for the knee and/or other joints with fixed or mobile bearing (made from polyethylene or other materials that are biocompatible) and that can adapt to the direction and type of forces received, and thus progressively absorb by viscoelastic behavior and redistribute the load forces, with a possibly improved range of joint movement.

Finally, to make use of synovial fluid for better lubrication of the endoprosthesis, and to have the ability to filter the wear debris which is produced by frictional forces of the materials.

In order for this invention to be achieved, compressible, resilient, adaptable, elastic materials, some of which will allow fluid and gases to penetrate, or a mechanical device or devices with spring action properties, or combination of these, will be inserted inbetween the mobile or fixed bearing body or inbetween the superior surface of the metal tibial component and the inferior surface of the bearing, or inbetween the tibial component body. In the case of the mobile bearing, this can only be achieved by using a mechanical device with spring action properties inbetween the superior part and inferior part of the mobile bearing. These inserted materials or mechanical devices are biocompatible, and their form and manufactured shape creates integral chambers, or are made to receive independent chambers, that communicate with the joint space through small holes or small openings on the fixed or mobile bearing body, the interposed material, or the tibial component. These chambers function as pumps through which the synovial fluid circulates in and out. The communicating chambers enclose biocompatible materials which in a rightful position allow synovial fluid and gases to penetrate, and as a result of this they filter the wear debris, polyethylene or metal, which is within the joint.

The invention will be described in relation to numbered designs in the form of a few examples, to avoid mentioning too many.

Design 1 shows in an illustrated form the function of lubrication and viscoelastic compression during loading.

Design 2 shows in an illustrated form the function of lubrication and viscoelastic decompression during unloading.

Design 3 shows in an illustrated form the function of lubrication and filtering during loading.

Design 4 shows a ground plan of an example in use according to the invention

Design 5 shows a section of level II-II of Design 4.

Design 6 shows a perspective form of another way of using of the invention in part form.

Design 7 shows a ground plan of design 6, of the way of use of the invention as Design 6.

Design 8 shows a section at the level of III-III of the Design 7.

Design 9 shows a section that describes another type of use of the invention.

Design 10 shows a section of another example of use according to the invention.

Design 11 shows a perspective view of an example of use, based on the invention using a mobile bearing.

Design 12 shows a section at the level of IV of Design 11 in relation to the mobile bearing (not in relation to the tibial component).

### DESCRIPTION IN DETAIL OF THE ABOVE DESIGNS

Designs 1,2,3 describe the basic principle and function of this invention and demonstrate the cycle of loading and unloading (for example during walking) according to the invention. The three designs show that a part of the fixed bearing (25) that articulates conformingly with the femoral component (15), the tibial component (24), and a compressible component (13) (according to the previous descriptions), which is inbetween the fixed bearing (25) and the tibial component (24). This fixed bearing (25), has hole (26), and this hole opens on the superior surface of the fixed bearing (25), where the femoral condyle (15) articulates with the fixed bearing (25), (as is shown in these diagrams, for descriptive reasons, only in relation to one condyle instead of two). The inferior opening of the hole (26) (distally) ends in a shaped chamber (14), which is formed of the wall of the superior surface of the tibial component (24), and the inferior surface of the fixed bearing (25), and circumferentially from the wall of the compressible material (13), of which the shape underneath the bottom of the hole (26), forms the chamber (14).

During the period when the endoprosthesis is not in use (for example sitting) the synovial fluid progressively collects in the concave part of the fixed bearing (25), and from there into the hole (26), to the chamber (14). During loading of the endoprosthesis (stance phase and walking), the vertical loading that is exerted by the femoral component (15), to the fixed bearing (25) is transmitted to the compressible component (13), the thickness of each, is reduced (13a) and the distance between the inferior surface of the fixed bearing (25), and the superior surface of the tibial component (24), becomes smaller (13a), and therefore there is a reduction in size and capacity of the chamber (14). As a result of the reduction in size and capacity of the chamber (14), the synovial fluid and gases which are collected in the chamber (14) are forced because of increased pressure to exit (16), through the hole (26), in the superior surface of the fixed bearing (25), of the part that articulates with the femoral component (15). The force and pressure of the synovial fluid spurts and exits (16) of the hole (26), or the chamber (14), is related to the amount of loading exerted upon the endoprosthesis. The presence of synovial fluid during loading, with some pressure and in a certain amount of it, at the fixed bearing (25) part which has better contact with the moving femoral component (15), reduces the frictional forces and wear between the contact surfaces of fixed bearing (25) and femoral component (15), because an amount of the synovial fluid interpolates (17) between the contact surfaces of the femoral component (15) and the fixed bearing (25), and the reduction of friction is mainly due to hydrostatic and hydrodynamic lubrication mechanism, and in some part of the lubrication with the squeeze-film mechanism (Basic Biomechanics of the Skeletal System, Victor H Frankel, Margareta Nordin, Lea & Febiger, Phil. USA.)

The synovial fluid and the gases which are accumulated in the chamber (14), need time to exit (16) from the hole (26), and this depends on the size of the hole (26), if it is covered by the femoral component (15), the viscosity of the synovial fluid and the amount of loading. Therefore the compressibility of the elastic component (13), and all this unit, depends also on the speed of the exit (16) of the synovial fluid and gases from the chamber (14), and this unit reacts not only in relation to the amount of loading but also to the time it receives the load. This reaction to the load is called viscoelasticity, and makes the endoprosthesis behave and absorb the forces in a similar way to a normal cartilaginous joint. It is this property of the invention that gives it the ability to receive an increase in the loading of the endoprosthesis, because it can absorb gradually and smoothly the forces by the compressible component (13) and also by the energy that is absorbed during exit (16)-entry (19) of the synovial fluid and gases from chamber (14). The re-entry (19) of synovial fluid and gases in the chamber (14), occurs during the phase of unloading of the endoprosthesis, design 2, and enlarges as the compressible component (13) resumes its previous size (13b) and the chamber (14) increase its size and capacity. At that moment the reduced pressure in chamber (14), in relation to the articular space, forces the synovial fluid and gases to re-enter (19) the chamber (14). The synovial fluid is directed to hole (26) because of the gravity, particularly as the superior opening of hole (26) is at the deepest point of the superior surface of the fixed bearing (25), the point of the largest contact surface area with femoral component (15). During the re-entry phase (19) of the synovial fluid and gases to the chamber (14), all the unit does not return immediately to the neutral position after unloading, because the re-entry phase takes some time for this, so the endoprosthesis keeps the viscoelastic behavior during unloading. Once they collect again in chamber (14) during the next loading phase, the cycle that has previously been described above starts again. During this cycle of compression (13a) and decompression (13b) of the fixed bearing (25) in relation to the tibial component (24), and exit (16)-entry (19) of the synovial fluid, interpolate in its path, design 3, a biocompatible, elastic, hydrophilic, or not hydrophilic material (21) that is permeable (22),(23) by the synovial fluid, which is resilient to pollution from stagnant proteins and/or other biological components of the synovial fluid, and is at the same time able to withhold debris, particles of metal and polyethylene or other materials produced by the friction of the parts of the endoprosthesis.

This invention therefore has the ability and the capability to filter and retain debris, reduce their quantity, and thus increase the lifespan of the endoprosthesis by avoiding osteolysis. The synovial fluid can pass (22),(23) entirely or as substrate through the filter. The appropriate materials (21) for filters can have as constituent parts, silicone, rubber, hydroxyl-ethyl-methacrylate(hema), the polyvinylpirrolidone (PVP), methylmethacrylate (A), or other materials known by commercial names such as ES70, manufactured by Essilor, or Balafilcon by Bausch and Lomp. Some of the above materials have elastic properties, others only hydrophilic, some elastic and hydrophilic, some are neither elastic nor hydrophilic but are only permeable to the synovial fluid, and their combined use can provide a material (21) suitable for filters. These named materials are not the only ones that can be used. There are several others that are suitable. As well as some forms shown in designs 6,7,8, the compressible material functions also as a filter, and the physical properties of the material in this form can be characterized by its ability to be compressible material and filter together, as compared to other materials that can only be used as filters. Following a detailed explanation in designs 4 and 5, an example is given of the way that this invention is used, where the fixed bearing (25) and the tibial component (24), use elastic and compressible material (13) placed inbetween them transversely and peripherally. This material (13) can be biocompatible using silicone, silastic or a form of rubber or other material. The peripheral compressible material (13), when in place creates a closed chamber (14) where its superior surface is the inferior surface of the fixed bearing (25), its inferior surface is the superior surface of the tibial component (24), while peripherally it is composed of compressible material (13). At the central area of the chamber (14) is positioned a filter material (21) which has the characteristic properties similar to those described in design 3 as filter material (21) properties. The areas of chamber (14) that are beneath the level of the contact surfaces of the fixed bearing (25) articulates with the femoral component (not shown), which remains empty, whilst in these areas holes (26) go through the body of the fixed bearing (25), bringing into contact the joint space and the superior surface of the fixed bearing (25), with the chamber (14). Another hole (27), transverses the fixed bearing (25), at a central point bringing in contact the area of the chamber (14) which contains the filter (21) with the joint space. A ring (28) with spring action properties made from biocompatible material stabilizes the fixed bearing (25) on the tibial component (24), allowing the fixed bearing (25) when it receives loads, to compress the elastic compressible material (13), and move nearer to the tibial component (24), and when unloading it returns to its initial position moving away from the tibial component (24), not being able to dislocate the fixed bearing (25) from the tibial component (24). The ring (28), may not cover all of the periphery of the tibial component (24), leaving a small space (28A), and having the ability to constrict, bringing its edges closer. Because of the constriction ability of the ring (28), the fixed bearing (25) can be coupled and decoupled and remains stable on the tibial component (24), whilst at the same time is compressible in relation to the tibial component (24). The shape and moulding of the ring (28), serves the above needs. The way that this type of invention works follows the descriptions of designs 1, 2, 3. The synovial fluid in this type of use the invention passes partially through the filter (21), and all the unit behaves both elastically and viscoelastically.

Designs 6,7, and 8, show another way of using the invention where the basic components remain the same as those in designs 4 and 5.

Chamber (14) is made out of the inferior part of the fixed bearing (25) the superior surface of the tibial component (24), and peripherally by the ring (28) of the stabilization-connection. In between the tibial component (24), and the fixed bearing (25), there is an elastic material (21), which is biocompatible and compressible, permeable to the synovial fluid, with all the characteristics described in design 3 as a filtering material (21). The holes (26), which communicate the chamber (14) with the joint space, have their superior opening on the superior surface of the fixed bearing (25) at the contact area with the femoral component (design not shown) and their inferior opening in direct contact with the compressible material (21).

This use of the invention follows the function described in designs 1,2,3, but it has the characteristic that most of the synovial fluid that enters (19) and then exits (16) during use, passes through the body of compressible material (21), mainly during the entry (19) to chamber (14) but also during exit (16). A small quantity enters the chamber (14) through the specially formed ring (28), as well as the small space (28A), and from the declivity of the circumferential margin (24A) of the tibial component. This creates a better filtering of debris. The whole unit has viscoelastic behavior considering the way that the endoprosthesis absorbs the forces exerted.

Designs 9,10, describe another way of use according to the invention where the basic components remain the same as designs 4,5,6,7,8, and with more analytical details , in design 9 the intermediate material (13) inbetween the fixed bearing (25) and the tibial component (24) is a biocompatible, compressible, elastic, non hydrophilic material (13) such as silicone, silastic, rubber or other material, which covers almost all the inferior surface of the fixed bearing (25) as shown in design 9, and as a result this endoprosthesis behaves as viscoelastic, but the elastic property is greater than the other designs described. In design 9, we have two as shown (with the possibility of additional) autonomous chambers (14) which partially take over space also within the interior part of the body of the fixed bearing (25) and within the body of compressible material (13). These chambers (14) communicate with the joint space with vertical holes (26) which traverse the body of the fixed bearing (25) in the areas of contact with the femoral condyles (not shown). In their superior part there is a noncompressible, non elastic, permeable by synovial fluid filter (21) with the properties similar to those described in design 3, as filter material (21) properties.

A spiral form (29) holds the filter (21) in the top part of chamber (14) whilst simultaneously permitting the passage of synovial fluid through the spiral form (29), and through the filter material (21). The bottom part of chamber (14) remains empty and the filter (21) does not come into contact with the tibial component (24) even during maximum compressive activity. The spiral form (29) (spring like) can be made out of any biocompatible material is capable of such a function. Functionally this way of using this invention is different, because filter (21) can be used without being compressible, and the small size of the chambers (14) and the specific placement that they have, create conditions of increased exit pressure for synovial fluid and improved lubrication on the basis of hydrostatic mechanism.

In design 10 there is a way of using the invention which behaves viscoelastically, having compressible material (13) permeable only to gases, having in its substance microchambers that work as the chambers (14) previously described, allowing this compressible material (13) to have the possibility to aspirate and push out gases during decompression and compression of the endoprosthesis through the hole (27A) and circumferential at the point of contact between the ring (28) and the fixed bearing (25). In this design the microchambers are not shown. Also in design 10 the compressible material (13) could be totally elastic if what is required is only elastic absorption of forces.

Designs 11,12 show, according to the invention, a way of using an endoprosthesis with a mobile bearing. The mobile bearing according to the designs consists of a superior part (30) an inferior part (31) and an intermediate part made out of a compressible mechanism (32) (for example a spring). These three parts are joined to each other by mechanical means, or biocompatible adhesive or by a combination of these, to a unified functioning mobile bearing that can move, glide, and rotate in all directions on the tibial component (24). These three parts (30),(31),(32) of the mobile bearing which are joined together, however varying the manufacturing characteristics (33) may be or have articulating surfaces (33) conforming or nonconforming, and using bollards or stops to prevent dislocation, allows it to work as a complete entity as far as compressibility of the mobile bearing is concerned and moving on the tibial component (24) as with, all other noncompressible mobile bearings already in use, but which lack the compressible element described.

The superior part (30) of the mobile bearing has vertical holes (26) that traverse its body and go through the superior part of compressible mechanism (32) into the chambers (14) placed in the empty spaces of the compressible mechanism (32). Also other holes (34) which have as a superior opening the superior surface of the superior part (30) of the mobile bearing, and inferior opening the inferior surface of the inferior part (31) of the mobile bearing, traversing through the intermediate compressible mechanism(32), leaving the synovial fluid to pass through to the contact surface area of the tibial component (24), with the inferior surface of the mobile bearing improving also the lubrication at this level. The ability of the mobile bearing to be compressed and also to receive or to create chambers (14) which communicate with its superior part (30) where the femoral component ( not shown) articulates, allowing the mobile bearing to function as described with this invention in a similar way as the endoprostheses with a fixed bearing (25) described previously.

It also could have empty spaces instead of chambers (14), with filter material (21) as described in the designs 6,7,8 or any other combination based on the ideas and the purpose of this invention. Also in relation to the suitable materials , the fixed bearing as well as the superior and inferior part of the mobile bearing, can be made out of ultra high molecular weight polyethylene, or from any biocompatible material appropriate for this use, such as carbon fibres, cobalt chromium alloy, alumina ceramic or zirconium, or a combination of them, or any other material that will give increased resilience with less thickness and therefore wider and more effective use of the invention. The use of the above mentioned materials or a combination can be used and in the case of the tibial component, or generally the components covering the articular surface of the bones, (which are usually made out of cobalt chromium alloy), and also for the compressible mechanisms (e.g. spring or ring) that can be used in the mobile bearing or inbetween the fixed bearing and the tibial component or as tibial component and fixed bearing connection ring. Specifically for the construction of a spring mechanism, any biocompatible metal or plastic or combination of these that can give resistance to repeated distortions and elastic properties. The invention can also be widely used when applied to the already available endoprostheses in clinical use, by some changes made in the shape and moulding of the inferior part of the fixed bearing which in combination with construction of an appropriate ring or mechanism will keep and stabilize the fixed bearing in place upon the tibial component, for each type of endoprostheses, allowing compression and decompression of the fixed bearing in relation to the tibial component, and in general the complete function of the invention without dislocation of the fixed bearing from the tibial component. This can be done without changes or with some very small changes being made to those already available in clinical use tibial components for knee endoprosthesis, reducing the cost of manufacturing. The same applies (as described in designs 11,12.) and in the case of the mobile bearing but also for all types of endoprostheses that are described for other joints (other than knee) and which have in their constituent parts, components covering the articular surface of the bones and fixed bearings or mobile bearings for better articulation.

Advangages:

The advantages of an endoprosthesis that contains a fixed bearing or a mobile bearing such as described above according to the invention are multiple and are related to the way it is produced. If simply the fixed bearing and the tibial component have inbetween them a compressible elastic material (design 10), or a mechanism specially in the case of a mobile bearing, then the fixed bearing and by extension the endoprosthesis has the ability to absorb and redistribute the forces that are exerted during use, with better and more homogeneous distribution upon the tibial and femoral component. In addition to the above benefits, full use of them is made when the ability of the fixed bearing, or of the mobile bearing, to be compressed and decompressed by itself or in relation to the tibial component is incorporated with materials and other forms that have been described for the function of self-lubrication, filtering, viscoelastic absorption of the forces and adaptation. The adaptation of the fixed bearing and the mobile bearing to the loads ensures a more conforming contact surface area with the femoral component. The improved lubrication that occurs on the contact surface area reduces friction and concurrently the wear of the mobile parts. Reduced wear means a longer lifespan of the endoprosthesis and reduction in wear debris in the joint space. The reduction of the wear debris is helped by the function of the filtering significantly reduces the probability of osteolysis, and in general the toxic effect that these particles which might have upon the joint and the body. The progressive absorption means that if after the exertion of a vertical load upon the joint another vertical load added to the first one, the endoproshesis still has the ability to absorb the first and all subsequent loads successfully as the compression range continues. For example on climbing or coming down slowly a step the gradual absorption allows the endoprosthesis to distribute and absorb the forces that are placed upon it through the range of movement rather than just the initial phase. The adaptability of the fixed bearing or mobile bearing according to the invention adds another significant advantage, for example when the fixed bearing receives forces at a specific point and for a prolonged period of time distributes and disperses the synovial fluid into the compressible material (e.g. designs 6,7,8) depending on to the direction of the forces that it receives. The adaptability allows the fixed bearing to change position in relation to the tibial component, a change in position that is not lost immediately after these forces or loads stop. If the same loading is repeated again immediately after the previous, the fixed bearing keeps the adapted position improving the mobility. This can also occur during deep sitting where the fixed bearing will acquire a posterior inclination, improving the range of movement of the endoprosthesis in flexion, improving its stability, and contributes a improved contact area with the femoral component. In fast walking where the knee hyperextends a viscoelastic adaptation of the fixed bearing in anterior inclination improves the extension of the joint and as result the gait. In fast walking the above described occur in the same direction in shorter periods of time and the viscoelastic behavior does not allow the fixed bearing to lose its anterior inclination. All these descriptions also apply to the mobile bearing.

The gradual absorption of the load, prolongs the time that synovial fluid exits during self-lubrication and maintains the lubrication mechanism, even in slow motion with greater loads (e.g. climbing stairs).

In total, the application of the invention in an endoprosthesis, allows it to receive larger loads, with less wear. An endoprosthesis that simulates a natural joint regarding function, an endoprosthesis that can be used in younger and more active patients, tolerating the increased activities, and prolonging its life expectancy. The possibilities of adaptation that it offers, make it easier for surgical application. In unicompartimental endoprosthesis, which is more sensitive to the forces applied than total knee endoprosthesis and achieves a more normal function because of more synovial fluid available, the use of this invention as a unicompartimental endoprosthesis is strongly recommended because of its ability to make more use of the synovial fluid. The descriptions of this invention and its use were only given in the form of examples in order to explain the concept, and its application in clinical use. Many changes and variations can be mode or added following the general principles of this invention. For example, the holes for the passage of the synovial fluid can be one or several, or one-way valves can be used, so that the synovial flow, is directed by certain holes only (e.g. Only through the filters), or the chambers as many as necessary can be an integral part of the bearing body or the tibial component substance. For this reason, this application has the intention of covering any variations, use or adaptation using the general principles and concept of this invention, and which fall within the limits of the appended claims.

1. Endoprosthesis of the knee joint with possibilities of viscoelastic absorption of forces, improved self-lubrication mechanisms and filtering of wear debris, which consists of a fixed bearing (25) that is held firmly with a tibial component (24), having an articular superior bearing surface for engagement with a femoral component (15), and is characterized by the fact that the inferior surface of the said fixed bearing (25) does not abut against the superior surface of the said tibial component (24), but inbetween them there is an interspace with a compressible mechanism (32) placed in a tranverse level, and the stabilization of the said fixed bearing (25) on the said tibial component (24) occurs with a circumferential connecting ring (28) or other mechanical means in such a way that it allows the fixed bearing (25) the possibility when it receives load or weight to compress the compressible mechanism (32), and the body of the fixed bearing (25) moves approaching the tibial component (24) making their interspace smaller (13a), and during decompression the body of the fixed bearing (25) moves to the opposite direction increasing (13b) the interspace between the said fixed bearing (25) and the said tibial component (24), and this movement of compression (13a) and decompression (13b) of the fixed bearing (25) to the tibial component (24) during use occurs with no possibility for decoupling or dislocation, of the said fixed bearing (25) from the said tibial component (24), and which compressible mechanisms (32) with their position, shape, and manufacturing, creates in combination with the inferior surface of the fixed bearing (25), with the tibial component (24), and with the connecting mechanical means and ring (28), one or more chambers (14), or receives one or more autonomous shaped molded chambers (14), which can change their size and capacity, said chambers (14) which communicate with the joint space through holes (26), and/or small openings (28A), said holes (26) which traverse through the fixed bearing (25) body, and their superior opening is at the superior surface of the fixed bearing (25), at areas where the synovial fluid is collected, the moving parts transmit the largest load, and where there is better conformity and articulation with the femoral component (15), said chambers (14) which aspirate and accumulate the synovial fluid and gases during unloading of the endoprosthesis, and exit (16) the fluid and gases to the joint space during loading, adding hydrostatic self-lubricated mechanism, and in this synovial fluid flow (16)-(19) of entry (16) and exit (19), from and to the chambers, interpolates appropriate materials (21) which are permeable (22),(23) to the synovial fluid, and filter the synovial fluid trapping the wear debris produced by the friction of the parts of the endoprosthesis, which are in the synovial fluid.

2. Endoprothesis of claim 1, which is characterized by the fact that inbetween the fixed bearing (25) and the tibial component (24), in the interspace, there are compressible materials (13) or a combination of compressible materials (13) and mechanisms (32), which said compressible material (13) and said compressible mechanisms (32) when placed, shaped, and manufactured, can create in combination with the inferior surface of the fixed bearing (25), with the tibial component (24) and with the connecting mechanical means or ring (28), one or more chambers (14), or receives one or more autonomous chambers related to the molding, and said compressible materials (13) which could be elastic with or without hydrophilic properties, permeable (22)(23) by synovial fluid, or to consist solely of elastic parts such as silicone or rubber non-permeable by synovial fluid, with parts permeable (22)(23) by synovial fluid, or to be elastic permeable by gases, or to be solely elastic parts non-permeable by gases with parts permeable by gases, said compressible materials (13) when permeable by synovial fluid function simultaneously as filter material (21) for trapping the wear debris which is in the synovial fluid.

3. Endoprothesis of claim 1, which has inbetween the fixed bearing (25) and the tibial component (24) in their interspace a compressible material (13), and is characterized by the fact that the said compressible material (13) has in its substance microchambers into which gases enter (16) during unloading and gases exit (19) during loading.

4. Endoprosthesis of the knee of claim 1 which is characterized by the fact that inbetween the fixed bearing (25) and the tibial component (24) in their interspace there is only compressible material (13) which is solely elastic.

5. Endoprosthesis of the knee of claim 1 characterized by the fact that instead of fixed bearing (25), a mobile bearing is used.

6. Endoprosthesis of the knee of claim 1, claim 3, and claim 5, which is characterized by the fact that it has compressible mechanism (32) inbetween and transversely through the body of the mobile bearing in its cross-section, or inbetween and transversely of the body of the fixed bearing (25) in its cross-section, or inbetween and transversely of the body of the tibial component (24) in its cross-section, where the superior part (30) of the body of said mobile bearing, or of said fixed bearing (25), or of said tibial component (24), the said inclusive compressible mechanism (32), and the inferior part (31) of the body of said mobile bearing, or of said fixed bearing (25), or of said tibial component (24) corresponding parts, are joined to each other to a unified compressible structure, and this unified compressible structure has one or more holes (26) which pass through its body to connect the chambers (14) with the joint space, said unified compressible structure which has the manufacturing characteristics (33), the articulating surfaces (33) or stops, in such a way that allows the unified compressible mobile bearing, or the unified compressible fixed bearing (25), or the unified compressible tibial component (24) to work and function as all other noncompressible mobile bearings, or noncompressible fixed bearings (25), or non compressible tibial component (24) already in use, and in addition for the compressible mobile bearing to move on the tibial component (24) as with all other noncompressible mobile bearings in use.

7. Enthoprosthesis of the knee joint of claim 1, claim 2, claim 5, and claim 6, which is characterized by the fact that the compressible mechanisms (32)(29) and connecting ring (28), have spring action properties and could be made from any metal, plastic, ceramic, alloy, or any combination of the above, or other material suitable and biocompatible for this use.

8. Enthoprosthesis of the knee joint of claim 1, claim 2, claim 3, claim 5, and claim 6, which is characterized by the fact that enclose one or more chambers (14) said chambers (14) which can adapt to different size and capacity, said chambers (14) which can be autonomous or can be an integral part of the knee components, said chambers (14) which may or may not have a filter (21) material inside, said chambers (14) which can be placed into the body of the fixed bearing (25) or the mobile bearing, said chambers (14) which can be placed into the body of the compressible material (13) and mechanism (32), said chambers (14) which can be placed into the body of the tibial component (24) or the component which covers the articular surface of the bone, said chambers (14) which communicate with the joint space through holes (26) and small openings (28A), and their capacity and size increase during unloading of the knee joint, and decrease during loading of the knee joint.

9. Enthoprosthesis of the knee joint as claimed in claim 1, claim 2, claim 3, claim 5, claim 6, and claim 8, in which the pressure into the chambers (14) can change by increasing or decreasing during loading and unloading of the joint, said pressure into the chambers (14) which increases or decreases in relation to the joint space pressure, and forces the synovial fluid and gases to pass through the holes (26) and small openings (28A) from the joint space to the chambers (14), and from the chambers (14) to the joint space, said passage of the synovial fluid and gases through the holes (26) and small openings (28A) create a viscoelastic behaviour during loading and unloading of the joint.

10. Enthoprosthesis of the knee joint as claimed in claim 1, claim 2, claim 5, claim 6, claim 8, and claim 9, in which the trapping of the wear debris, and filtering of the synovial fluid occur in a mechanical way from the filter (21) materials which interpolate to the synovial fluid flow (16)-(19), said synovial fluid flow which created by the movement of the fluid from the chambers (14) to the joint space and from the joint space to the chambers (14), said filter (21) materials which are elastic or not elastic, biocompatible, hydrophilic or not hydrophilic, permeable by the synovial fluid, resilient to biological components of the joint fluid, said filter (21) materials could have as constituent parts silicone, rubber, hydroxyethylmethacrylate(hema), polyvinylprirrolidone(PVP), methylmethacrylate (MMA), or any other appropriate materials with suitable mechanical properties, and can be used for filtering the synovial fluid from wear debris.

11. Endoprosthesis of the knee joint as claimed in claim 1, claim 2, claim 5, claim 6, claim 8, claim 9, and claim 10, in which the superior openings of some or all of the holes (26) of the bearings, are at the concave superior surface of the fixed (25) or mobile bearing, where the femoral component (15) has better articulation, transmits larger loads, and more synovial fluid surfaces, said superior openings of the holes (26) from which spurt out (19) with pressure the synovial fluid augmenting the already normal lubrication mechanisms of the joint, and creating hydrostatic lubrication mechanisms.

12. Endoprosthesis of the knee joint as claimed in claim 5, claim 6, and claim 11, which is characterized by the fact that it has a mobile bearing which has one or more holes (34) which traverse vertically the body of the mobile bearing, said holes (34) which have as a superior opening the superior surface of the mobile bearing and as a inferior opening the inferior surface of the mobile bearing, allowing the synovial fluid to pass through, into the openings and holes (34) from the superior surface of the mobile bearing to the contact surface area of the tibial component (24) with the inferior surface of the mobile bearing.

13. Endoprosthesis of the knee joint as claimed in any one of the preceding claims for unicompartmental endoprosthesis.

14. Endoprosthesis as claimed in claim 1, claim 3, claim 7, claim 8, claim 9, claim 10, and claim 11, for hip joint, and for ankle joint.

15. Endoprosthesis of the knee joint as claimed in any one of the preceding claims in which their constituent parts are biocompatible, and can be manufactured from any plastic, metal, ceramic, alloy or other appropriate materials.
